(19) United States
(12) Patent Application Publication
(10) Pub. No.: US 2026/0097159 A1
(43) Pub. Date: Apr. 9, 2026

(54) SYSTEMS AND METHODS OF SAFE MODEL-BASED MULTIVARIABLE CONTROL OF PERITONEAL PERFUSION

(71) Applicant: University of Maryland, College Park, College Park, MD (US)

(72) Inventors: Yejin MOON, Olney, MD (US); Hosam K. FATHY, Kensington, MD (US)

(21) Appl. No.: 19/348,083

(22) Filed: Oct. 2, 2025

Related U.S. Application Data
(60) Provisional application No. 63/703,148, filed on Oct. 3, 2024.

(51) Int. Cl. A61M 1/28; A61M 60/113; A61M 60/279; A61M 60/37; A61M 60/546; G16H 20/17

(52) U.S. Cl. CPC A61M 1/282; A61M 1/281; A61M 60/113; A61M 60/279; A61M 60/37; A61M 60/546; G16H 20/17; A61M 2205/3334; A61M 2205/3393

(57) ABSTRACT

In accordance with the present disclosure, a method for automatically controlled peritoneal perfusion includes receiving perfusion parameters such as a predefined intra-abdominal volume and inflow rate and circulating a perfusate between a reservoir and a subject's cavity using an inflow pump and an outflow pump. A reservoir volume may be measured by a weight sensor, inflow data may be obtained from a flow-rate sensor and a commanded flow rate may be accessed from the outflow pump. A drainage efficiency of the outflow pump may then be estimated using the reservoir volume, inflow rate, and the commanded outflow rate. A drainage efficiency model may be identified from experimental or operational data to represent efficiency dynamics. Based on this model, a perfusion safety control routine may evaluate a safety barrier function and adjust operation of the inflow and outflow pumps to maintain safety and compliance with the perfusion parameters.

### CROSS-REFERENCE TO RELATED APPLICATION AND CLAIM OF PRIORITY

This application claims the benefit of, and priority to, U.S. Provisional Patent Application No. 63/703,148, filed on Oct. 3, 2024, the entire contents of which are hereby incorporated herein by reference.

### GOVERNMENT LICENSE RIGHTS

This invention was made with government support under CMMI2031251 awarded by the National Science Foundation. The government has certain rights in the invention.

### TECHNICAL FIELD

The present disclosure relates generally to the field of medical perfusion systems and control engineering. More specifically, the present disclosure provides systems, devices, and methods for automated and safe regulation of peritoneal perfusion using model-based multivariable control techniques to balance inflow, outflow, and safety constraints during therapeutic interventions.

### BACKGROUND

Peritoneal perfusion, in which fluid is circulated or dwelled within a patient's abdominal cavity, has been investigated for a variety of clinical applications. Examples include peritoneal dialysis for toxin clearance, hyperthermic chemotherapy for localized cancer treatment, and emerging methods for oxygenation using oxygenated perfluorocarbons (PFCs). The latter approach has the potential to serve as an extrapulmonary means of gas exchange, providing supplemental oxygenation for patients suffering from respiratory insufficiency, such as acute respiratory distress syndrome (ARDS) or viral pneumonia.

Despite these potential benefits, peritoneal perfusion presents significant safety challenges. One risk arises from excessive intra-abdominal volume (IAV), which directly increases intra-abdominal pressure (IAP). Elevated IAP can progress to intra-abdominal hypertension (IAH) and, in severe or prolonged cases, abdominal compartment syndrome (ACS), a life-threatening condition that can cause multi-organ failure. A second risk is associated with fluid drainage. Excessive suction pressure applied at the drainage cannula can induce tissue trauma, serosal injury, or cavitation, while partial or complete occlusion of drainage outflow can lead to discrepancies between commanded and actual flowrates, undermining volume control and increasing the risk of IAH.

Conventional approaches to peritoneal perfusion often rely on manual adjustment of pump flowrates or fixed treatment protocols. These approaches are limited in their ability to manage the complex tradeoffs between inflow, outflow, and safety. For example, reducing suction pressure may protect tissue but compromises drainage, whereas increasing suction improves drainage but heightens the risk of injury and occlusion. Existing systems also generally lack real-time monitoring and adaptive control mechanisms capable of simultaneously addressing these competing risks.

Therefore, there remains a need for automated, compact, and adaptive peritoneal perfusion systems that can monitor drainage efficiency, detect outflow occlusions, and dynamically regulate inflow and outflow pump operation.

### SUMMARY

In accordance with the present disclosure, a method for automatically controlled peritoneal perfusion includes receiving perfusion parameters including at least a predefined intra-abdominal volume and a predefined inflow rate, circulating a perfusate between a reservoir and a cavity of a subject using an inflow pump and an outflow pump, measuring a reservoir volume using a weight sensor positioned beneath the reservoir, generating inflow data using a flow-rate sensor coupled to the inflow pump, accessing a commanded outflow rate from the outflow pump, estimating a drainage efficiency of the outflow pump based on at least the reservoir volume, the inflow rate, and the commanded outflow rate, identifying a drainage efficiency model from experimental or operational data to represent efficiency dynamics, and applying a perfusion safety control routine that evaluates a safety barrier function based on the drainage efficiency model and adjusting circulation of perfusate between the inflow pump and the outflow pump based on the evaluated safety barrier function.

In an aspect, the method may include perfusion parameters having a minimum drainage efficiency threshold.

In an aspect, the method may include estimating the drainage efficiency by solving a least-squares regression with exponential forgetting to adaptively update the efficiency estimate over time.

In an aspect, the method may include identifying the drainage efficiency model by fitting a state-space representation of drainage efficiency dynamics to experimental or operational data.

In an aspect, the method may include a state-space model having an occlusion-related state variable governed by a linear differential equation with constant parameters.

In an aspect, the method may include applying the perfusion safety control routine by evaluating a safety barrier function that maintains drainage efficiency above the minimum drainage efficiency threshold.

In an aspect, the method may include applying the perfusion safety control routine by assuming the inflow pump rate equals a desired inflow rate, computing an unconstrained outflow pump command, determining whether the unconstrained pump commands satisfy the safety barrier function, and computing constrained pump commands that maintain safety when the unconstrained pump commands violate the safety barrier function.

In an aspect, the method may include measuring the reservoir volume by the weight cell disposed beneath the reservoir.

In an aspect, the method may include measuring the inflow data by the flow-rate sensor coupled to the inflow pump.

In an aspect, the method may include measuring the outflow data by the pressure sensor disposed along a return line of the outflow pump.

In an aspect, the method may include peritoneal perfusion as a hyperthermic intraperitoneal chemotherapy procedure, and the method may be performed during the procedure.

In accordance with the present disclosure, an automatically controlled peritoneal perfusion system includes a reservoir configured to hold a perfusate, an inflow pump fluidly coupled to the reservoir and configured to deliver the perfusate to a peritoneal cavity of a subject, an outflow pump fluidly coupled to the peritoneal cavity and configured to withdraw the perfusate and return the perfusate to the reservoir, a plurality of sensors including at least one weight sensor, at least one flow-rate sensor, and at least one pressure sensor, at least one processor, and at least one memory storing instructions which, when executed by the processor, cause the system to receive perfusion parameters including at least a target intra-abdominal volume and a target inflow rate, circulate the perfusate between the reservoir and the peritoneal cavity using the inflow pump and the outflow pump, measure a reservoir volume using the weight sensor, generate inflow data using the flow-rate sensor, access commended outflow rate from the outflow pump, estimate a drainage efficiency of the outflow pump based at least on the reservoir volume, the inflow data, and the commanded outflow rate, and apply a perfusion safety control routine that evaluates the estimated drainage efficiency relative to the perfusion parameters and adjusts operation of the inflow pump and the outflow pump based on the evaluation.

In an aspect, the system may include a processor configured to receive perfusion parameters including a minimum drainage efficiency threshold.

In an aspect, the system may include instructions that, when executed by the processor, may cause the system to estimate drainage efficiency by solving a least-squares regression with exponential forgetting to adaptively update the efficiency estimate over time.

In an aspect, the system may include instructions that, when executed by the processor, may cause the system to identify a drainage efficiency model by fitting a state-space representation of drainage efficiency dynamics to experimental or operational data.

In an aspect, the system may include a state-space model having an occlusion-related state variable governed by a linear differential equation with constant parameters.

In an aspect, the system may include instructions that, when executed by the processor, may cause the system to evaluate a safety barrier function that maintains drainage efficiency above the minimum drainage efficiency threshold.

In an aspect, the system may include instructions that, when executed by the processor, may cause the system to assume an inflow pump rate equal to a desired inflow rate, compute an unconstrained outflow pump command, determine whether the unconstrained pump commands satisfy the safety barrier function, and when the unconstrained pump commands violate the safety barrier function, compute constrained pump commands that maintain safety.

In an aspect, the system may include a weight sensor that may be a load cell disposed beneath the reservoir to measure reservoir volume.

In an aspect, the system may include a flow-rate sensor that may be coupled to the inflow pump to measure inflow data.

In an aspect, the system may include a pressure sensor that may be disposed along a return line of the outflow pump to measure outflow data.

In an aspect, the system may include a peritoneal perfusion system that may be a hyperthermic intraperitoneal chemotherapy system configured for use during a HIPEC procedure.

In accordance with the present disclosure, a non-transitory computer-readable medium stores instructions that, when executed by at least one processor, cause a system for automated peritoneal perfusion to receive perfusion parameters including at least a predefined intra-abdominal volume and a predefined inflow rate, circulate a perfusate between a reservoir and a cavity of a subject using an inflow pump and an outflow pump, measure a reservoir volume using a weight sensor positioned beneath the reservoir, generate inflow data using a flow-rate sensor coupled to the inflow pump, access commanded outflow rate from the outflow pump, estimate a drainage efficiency of the outflow pump based on at least the reservoir volume, the inflow rate, and a commanded outflow rate, identify a drainage efficiency model from experimental or operational data to represent efficiency dynamics, and apply a perfusion safety control routine that evaluates a safety barrier function based on the drainage efficiency model and adjusts circulation of perfusate between the inflow pump and the outflow pump based on the evaluated safety barrier function.

### BRIEF DESCRIPTION OF THE DRAWINGS

A better understanding of the features and advantages of the present disclosure will be obtained by reference to the following detailed description that sets forth illustrative embodiments, in which the principles of the present disclosure are utilized, and the accompanying drawings of which:

FIG. 1 illustrates a diagram of a system for automatic peritoneal perfusion control, in accordance with the present disclosure;

FIG. 2 illustrates a block diagram of a controller of the system of FIG. 1, in accordance with the present disclosure;

FIG. 3 illustrates a diagram of a perfusion flow pump system of the system of FIG. 1, in accordance with the present disclosure;

FIG. 4 is a flowchart of an exemplary method for controlling peritoneal perfusion by the perfusion flow pump system, in accordance with the present disclosure;

FIGS. 5A-5C are exemplary graphical illustrations of results of drainage efficiency estimation by the system of FIG. 1, in accordance with the present disclosure;

FIG. 6 is a graphical illustration of a comparison between experimental drainage efficiency and estimated drainage efficiency as a function of time identified by the system of FIG. 1, in accordance with the present disclosure;

FIGS. 7A and 7B are graphical illustrations of commanded flowrates for an inflow pump and an outflow pump of the system of FIG. 3, identified by the system of FIG. 1, in accordance with the present disclosure.

FIGS. 8A and 8B are a graphical illustration of the volume of perfusate in a canister and the drainage efficiency in a safety constrained control routine of the system of FIG. 1, in accordance with the present disclosure;

FIGS. 9A and 9B are graphical illustrations of commanded flowrates for an inflow pump and an outflow pump of the system of FIG. 3, in a safety constrained control routine of the system of FIG. 1, in accordance with the present disclosure; and

FIGS. 10A and 10B are graphical illustrations of the volume of perfusate in a canister and the drainage efficiency in a safety constrained control routine of the system of FIG. 1, in accordance with the present disclosure.

### DETAILED DESCRIPTION

The present disclosure relates generally to the field of provides systems, devices, and methods for automated and safe regulation of peritoneal perfusion using model-based multivariable control techniques to balance inflow, outflow, and safety constraints during therapeutic interventions.

Although the present disclosure will be described in terms of specific examples, it will be readily apparent to those skilled in this art that various modifications, rearrangements, and substitutions may be made without departing from the spirit of the present disclosure. The scope of the present disclosure is defined by the claims appended hereto.

For purposes of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

Unless expressly defined herein, all terms used in this disclosure, including the claims, are intended to have their ordinary and customary meaning as understood by a person of ordinary skill in the art to which this subject matter pertains. Where a term is expressly defined, that definition shall govern. Singular forms include plural forms unless the context clearly dictates otherwise, and “comprising” is used in a non-limiting, open-ended sense.

The term “perfusate” (P) refers to any liquid medium circulated through the peritoneal cavity of a subject, including but not limited to oxygenated perfluorocarbon (PFC), saline, chemotherapeutic agents, or combinations thereof. The terms perfluorocarbon (PFC) and perfusate may be used interchangeably throughout this disclosure.

The term “inflow pump” refers to any device or system configured to propel perfusate into a subject's cavity. Examples include peristaltic pumps, rotary pumps, positive displacement pumps, roller pumps, or diaphragm pumps.

The term “outflow pump” refers to any device or system configured to remove perfusate from a subject's cavity. Examples include peristaltic pumps, rotary pumps, vacuum-assisted suction pumps, or equivalents.

The term “weight sensor” refers to a device configured to measure the mass of reservoir 420 and its contents, thereby inferring perfusate volume. Examples include strain-gauge load cells, piezoelectric transducers, and capacitive sensors.

The term “flow-rate sensor” refers to a device configured to measure a volumetric flowrate of perfusate through a flow path. Examples include ultrasonic, turbine, Coriolis, differential-pressure, and thermal mass flow sensors.

The term “pressure sensor” refers to a device configured to measure absolute, gauge, or differential pressure in the main perfusion line. Examples include MEMS-based transducers, piezoelectric sensors, or manometric devices.

The term “intra-abdominal volume” (IAV) refers to the volume of perfusate retained within the peritoneal cavity of the subject, and “intra-abdominal pressure” (IAP) refers to the pressure associated with that retained volume.

The term “drainage efficiency” (θ) refers to the ratio of the true outflow rate from the peritoneal cavity to the commanded outflow rate of outflow pump 600, as estimated by controller 200 based on sensor feedback.

The term “occlusion” refers to a partial or complete blockage of fluid flow within the outflow pathway, including but not limited to obstruction of drainage cannulas, tissue collapse around cannula tips, suction-induced apposition of peritoneal surfaces, or cavitation within pump tubing. Occlusion may be transient or sustained and can result in decreased drainage efficiency, elevated intra-abdominal volume (IAV), or negative pressure-induced tissue trauma if not detected and corrected. As used herein, occlusion may be inferred from discrepancies between commanded and actual outflow performance, excessive suction pressure, or sudden changes in reservoir volume dynamics.

The term “controller” refers to a computing device comprising at least one processor and memory, configured to receive sensor signals, execute estimation and modeling routines, apply a safety barrier function, and command pump actuation in real time.

The present disclosure is generally related to systems and methods for automated peritoneal perfusion in medical procedures. More specifically, the present disclosure provides an integrated system and method for circulating a perfusate between an external reservoir and the peritoneal cavity of a subject while monitoring drainage efficiency, intra-abdominal volume (IAV), and intra-abdominal pressure (IAP). The disclosed system employs external sensors, including a weight sensor, flow-rate sensor, and pressure sensor, together with a controller that estimates drainage efficiency and applies a safety barrier function to dynamically regulate inflow and outflow pump operation. In this way, the system enables safe, real-time management of intra-abdominal fluid circulation without requiring invasive sensing within the body cavity.

The perfusion flow pump system disclosed herein is applicable to a variety of clinical and research settings requiring controlled fluid exchange between a fluid reservoir and an internal body cavity. In exemplary embodiments, the system may be used for hyperthermic intraperitoneal chemotherapy (HIPEC), peritoneal dialysis, peritoneal oxygenation, or other therapeutic modalities where circulation efficiency and safety are critical. Although examples herein primarily reference the peritoneal cavity of the abdomen, the system architecture and control methods are broadly applicable to other anatomical regions and extracorporeal configurations involving closed-loop fluid perfusion.

Conventional peritoneal perfusion systems generally rely on manual adjustment of pump parameters and intermittent clinical observation to regulate fluid balance and flowrate. These manual approaches are subject to significant limitations. For example, unregulated suction from the outflow pump may lead to partial or complete catheter occlusion, cavitation, or tissue trauma. Similarly, unmonitored inflow may result in the accumulation of excess fluid within the peritoneal cavity, leading to elevated intra-abdominal volume (IAV) and intra-abdominal pressure (IAP). IAV refers to the total volume of fluid retained within the peritoneal cavity, while IAP refers to the pressure exerted by this fluid on surrounding organs and tissues. Elevation of IAP beyond safe limits is associated with abdominal compartment syndrome, impaired organ perfusion, and other adverse outcomes. Existing systems may include alarms to notify clinicians of abnormal conditions but generally lack closed-loop control capable of automatically adjusting pump operation in real time to maintain safety.

The present disclosure addresses these shortcomings by introducing an automated, safety-constrained perfusion control system that integrates real-time estimation of drainage efficiency with a data-driven state-space model of occlusion dynamics. The system utilizes noninvasive external sensors, including a weight sensor, flow-rate sensor, and pressure sensor, to acquire continuous measurements of reservoir volume, inflow rate, and suction pressure, respectively. These measurements are used to estimate a drainage efficiency parameter (θ) that characterizes the effectiveness of fluid withdrawal from the cavity. Based on this estimate, the system identifies a predictive model of drainage dynamics and applies a safety barrier function that constrains pump commands when efficiency trends suggest emerging risk. The controller thereby enforces predefined safety thresholds during operation, without requiring invasive intra-abdominal instrumentation.

In this way, the system provides a technical solution to the technical problem of maintaining safe and effective peritoneal perfusion in the presence of variable physiological and procedural conditions. The disclosed control method combines least-squares estimation, state-space modeling, and real-time barrier-constrained control adjustment to achieve a dynamic balance between therapeutic efficacy and patient safety. Specifically, the system mitigates the risk of tissue injury from excessive suction, and the risk of organ dysfunction from perfusate over-delivery. While increasing suction pressure can accelerate drainage and reduce IAV and IAP, excessive suction may cause collapse of soft tissue, catheter blockage, or cavitation. Conversely, over-infusion of perfusate can elevate IAV and IAP to unsafe levels. By monitoring these dynamics continuously through external sensors and adjusting pump actuation accordingly, the system minimizes clinician burden, enhances treatment precision, and supports a range of clinical perfusion workflows in a closed-loop, autonomous fashion.

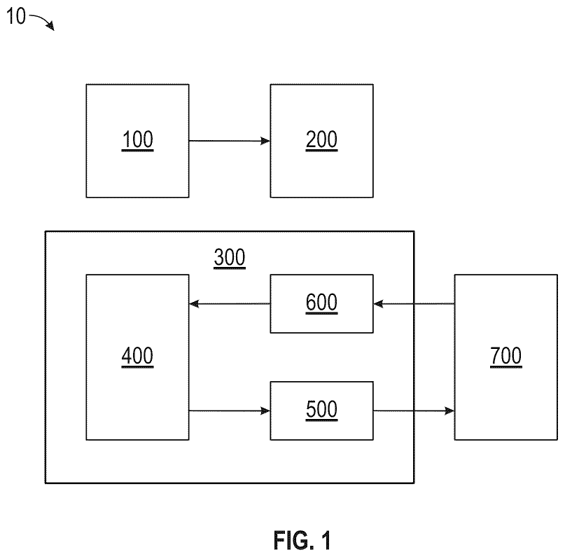

FIG. 1 illustrates an automatic peritoneal perfusion control system 10 in accordance with embodiments of the present disclosure. The system 10 includes an interface module 100, a controller 200, a perfusion flow pump system 300, and a subject 700. The interface module 100 is configured to receive one or more user-defined perfusion parameters, such as a desired inflow rate, a target intra-abdominal volume (IAV), and a minimum acceptable drainage efficiency. These inputs are communicated to the controller 200, which is configured to execute a model-based control algorithm to regulate fluid inflow and outflow during a peritoneal perfusion procedure.

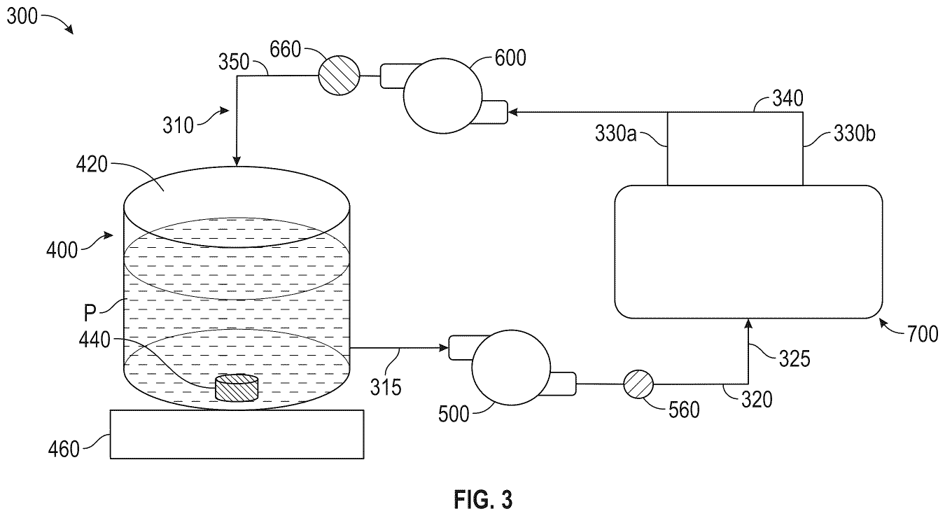

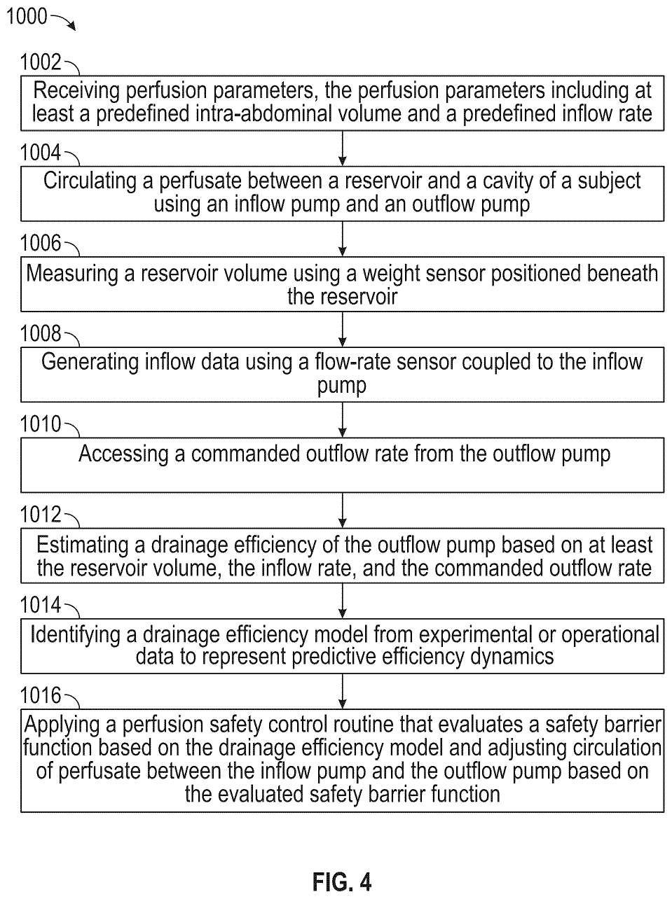

The perfusion flow pump system 300 includes a monitoring unit 400, an inflow pump 500, and an outflow pump 600. The monitoring unit 400 may include a reservoir in fluid communication with a weight sensor, enabling the system to estimate the net fluid volume delivered to or withdrawn from the subject 700. The controller 200 is operably coupled to both the inflow pump 500 and the outflow pump 600 and is configured to transmit control signals that govern each pump's flowrate based on safety constraints and the user-defined perfusion targets. The system 10 is described in more detail below with reference to FIGS. 3 and 4.

In some embodiments, the controller 200 continuously monitors estimated drainage efficiency, defined as the ratio between actual and commanded outflow. When drainage efficiency approaches or falls below a predefined threshold, the controller 200 may initiate a switching control strategy to mitigate potential safety risks, such as suction-induced tissue trauma or fluid retention. The interface module 100 may optionally include a graphical user interface (GUI), touch display, or software dashboard that allows a clinician to enter inputs, monitor system performance, and receive safety alerts related to perfusion efficiency, flowrate imbalances, or volume excursions.

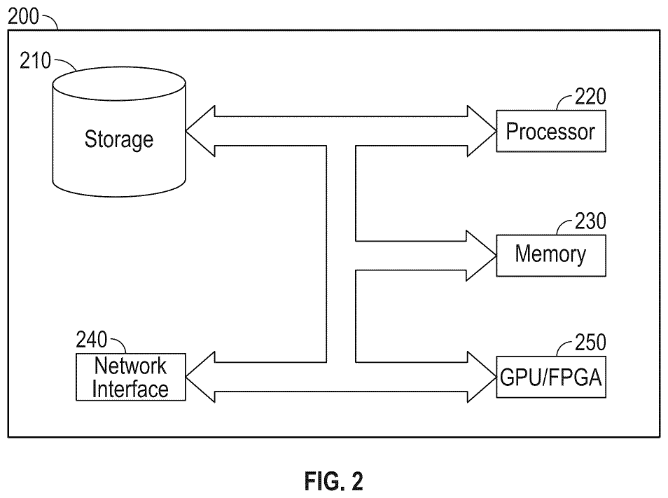

Referring now to FIG. 2, exemplary components in the controller 200 in accordance with aspects of the present disclosure include, for example, a database 210, one or more processors 220, at least one memory 230, and a network interface 240. In aspects, the controller 200 may include a graphical processing unit (GPU) 250, which may be used for processing machine learning models.

The processor 220 is configured to execute control instructions using data from the monitoring system 400 and inputs received via the interface module 100. The memory 230 may store transient data and state variables, while the storage module 210 may retain historical perfusion data and control parameters. In some embodiments, the network interface 240 enables communication with external systems for data logging or remote supervision. The controller 200 generates control signals for the inflow pump 500 and outflow pump 600 based on user-defined inputs and real-time system feedback. Under safe conditions, the controller 200 operates the pumps to achieve the desired perfusion targets. If drainage efficiency falls below a specified threshold, the controller may adjust pump flowrates to restore safe operation.

Database 210 can be located in storage. The term “storage” may refer to any device or material from which information may be capable of being accessed, reproduced, and/or held in a chemical-based digital form, or an electromagnetic or optical form for access by a computer processor. Storage may be, for example, volatile memory such as RAM, non-volatile memory, which permanently holds digital data until purposely erased, such as flash memory, magnetic devices such as hard disk drives, and optical media such as a CD, DVD, Blu-ray disc, or the like. In various embodiments, data may be stored on the controller 200, including, for example, user preferences, historical data, and/or other data. The data can be stored in database 210 and sent via the system bus to the processor 220.

As will be described in more detail later herein, the processor 220 executes various processes based on instructions that can be stored in the server memory 230 and utilizing the data from the database 210. The illustration of FIG. 2 is exemplary, and it will be understood by persons skilled in the art that other components may exist in a controller 200. Such other components are not illustrated in FIG. 2 for clarity of illustration.

FIG. 3 illustrates an embodiment of the perfusion flow pump system 300 in accordance with aspects of the present disclosure. The perfusion flow pump system 300 includes a monitoring unit 400, an inflow pump 500, an outflow pump 600, a main perfusion line 310, at least one perfusion cannula 325, and at least one drainage cannula 330a, 330b inserted into a cavity of a subject 700. The inflow pump 500 and outflow pump 600 are peristaltic pumps that may be operated in a reversible or variable-speed mode. In aspects, the inflow pump 500 delivers perfusate P to the subject 700 and outflow pump 600 applies suction pressure to drain perfusate P from the subject 700.

The system 300 further includes a plurality of flow lines, 315, 320, 340, and 350 that are fluidly coupled and form the main perfusion line 310. The main perfusion line 310 interconnects the monitoring unit 400, the inflow pump 500, the outflow pump 600, the at least one perfusion cannula 325, and the at least one drainage cannula 330a, 330b of the system 300 to form a closed-loop circuit. The cavity of subject 700 may be the peritoneal cavity in clinical contexts such as peritoneal dialysis, HIPEC, or peritoneal oxygenation. In an aspect, flow line 315 couples the reservoir 420 to the inflow pump 500 and flow line 320 (referred to as “inflow line”) couples the inflow pump 500 to the perfusion cannula 325, which communicates with the cavity of the subject 700. Flow line 340 fluidly couples drainage cannulas 330a, 330b to the outflow pump 600, and flow line 350 (referred to as “return line”) couples the outflow pump 600 back to the reservoir 420. In aspects, there may be only one drainage cannula, or a plurality of drainage cannuals. The flow lines may be implemented using medical-grade tubing formed from flexible polymeric materials such as silicone, polyurethane, or PVC.

The monitoring unit 400 includes a reservoir 420 configured to hold a perfusate P, such as oxygenated perfluorocarbons (PFC). The reservoir 420 is supported on weight sensor 460, which continuously measures the combined weight of the reservoir and its contents, thereby enabling calculation of the perfusate P volume. An oxygenator 440 may be positioned within or coupled to the reservoir 420 to enrich the perfusate P with oxygen prior to circulation. In this way, the monitoring unit 400 functions both as a storage chamber for perfusate P and as a source of real-time volume and conditioning data for the system 10.

In exemplary aspects, the perfusate P may comprise a physiologically compatible solution such as normal saline, lactated Ringer's solution, glucose-based dialysate, or an icodextrin-based solution. In certain therapeutic applications, the perfusate P may include chemotherapeutic agents dissolved in a carrier fluid for intraperitoneal delivery. Additional perfusates may include colloid-based solutions (e.g., albumin or hydroxyethyl starch), hemoglobin-based oxygen carriers, or other buffered oxygen-carrying emulsions. The specific composition of perfusate P may be selected based on the intended clinical use, such as peritoneal dialysis, oxygenation, drug delivery, or hyperthermic intraperitoneal chemotherapy (HIPEC). In some embodiments, the perfusate P may be heated or oxygenated prior to administration using inline heat exchangers or membrane oxygenators.

In an aspect, the system 300 further comprises a plurality of primary sensors positioned externally along the main perfusion line 310. A weight sensor 460 is disposed beneath the reservoir 420 and is configured to measure fluid mass of perfusate P and thereby calculate perfusate P volume over time. A flow-rate sensor 560 is coupled to inflow line 320 and is configured to monitor the delivery flowrate of perfusate P being supplied by inflow pump 500 through perfusion cannula 325 and into the cavity of subject 700. A pressure sensor 660 is disposed along return flow line 350 to measure suction pressure generated by outflow pump 600 during drainage through cannulas 330a, 330b. Together, the weight sensor 460, flow-rate sensor 560, and outflow pump 600 provide continuous real-time information regarding perfusate volume, inflow conditions, and commanded outflow rate, which allow the controller 200 to regulate intra-abdominal volume (IAV) and intra-abdominal pressure (IAP) without requiring additional sensors inside the cavity of the subject 700. This external sensor configuration provides a noninvasive means to balance fluid delivery and drainage, mitigating risks of over-infusion (excessive IAV/IAP) and over-suction (tissue trauma or catheter occlusion) without requiring intracorporeal sensors, allowing for external-only monitoring to ensure patient comfort and minimize risk of infection. Although not used for drainage efficiency estimation in the embodiments described herein, in exemplary aspects, the suction pressure measured by pressure sensor 660 may be used for safety monitoring and may be provided to the controller 200 for drainage efficiency and control.

In an aspect, weight sensor 460 may be a strain-gauge, piezoelectric, or capacitive load cell. Flow-rate sensor 560 may be ultrasonic, turbine, thermal mass, coriolis, or differential-pressure type. Pressure sensor 660 may be a MEMS-based absolute, gauge, or differential transducer. Each of the sensors may provide analog or digital output signals to controller 200 via wired or wireless data interfaces. In certain aspects, additional flow-rate or pressure sensors may be coupled to flow line 340 and/or flow line 320. In exemplary aspects, the number of sensors placed within the body cavity is minimized, such that all primary sensing is performed externally along the main perfusion line 310. In aspects, the sensors are sampled at a rate sufficient to support real-time estimation and control (e.g., 10-100 Hz), enabling dynamic tracking of flow conditions and perfusate balance. Furthermore, the inflow pump 500 and outflow pump 600 may be peristaltic pumps operating in variable-speed or reversible modes. In alternative embodiments, either or both pumps may be embodied as rotary pumps, diaphragm pumps, gear pumps, roller pumps, or vacuum-assisted suction devices. The system may be configured to operate under open-loop or closed-loop control depending on clinical objectives and safety constraints.

In operation of system 300, one or more perfusion cannulas 325 and drainage cannulas 330a, 330b are inserted into the peritoneal cavity of the subject 700. The perfusion cannulas 325 are configured to deliver perfusate P into the cavity, while the drainage cannulas 330a, 330b are configured to withdraw the fluid after dwell time. The cannulas are fluidly connected to the main perfusion line 310, which is formed by flow lines 315, 320, 340, and 350. The plurality of flow lines 315, 320, 340, and 350 are fluidly coupled to establish a closed-loop pathway that transfers perfusate P from reservoir 420 into the peritoneal cavity of subject 700 and returns the withdrawn perfusate back into reservoir 420. The circulation is dynamically regulated during operation, with inflow and outflow driven by inflow pump 500 and outflow pump 600, respectively. The system 10 continuously evaluates and modulates this flow in system 300 using control logic executed by controller 200 to meet operator-defined perfusion targets while maintaining safe pressure and volume conditions within the cavity.

More specifically, fluid exits reservoir 420 along flow line 315 and enters inflow pump 500. Inflow pump 500, in one embodiment a peristaltic pump, propels perfusate through flow line 320 and into perfusion cannula 325, which introduces fluid directly into the cavity of subject 700. Flow-rate sensor 560 positioned along flow line 320 continuously monitors the delivery flowrate of the perfusate, thereby confirming the commanded inflow rate and providing real-time feedback. As perfusate accumulates within the cavity, intra-abdominal volume (IAV) rises. Because the abdominal cavity is compliant but not rigid, increases in IAV proportionally elevate intra-abdominal pressure (IAP), which must remain within safe limits to avoid abdominal compartment syndrome. The controller 200 uses the measured inflow rate measured by the flow-rate sensor 560 in conjunction with the commanded outflow rate determined from the outflow pump 600 to estimate the net rate of fluid accumulation in the abdominal cavity, thereby inferring real-time changes in IAV without requiring direct measurement within the cavity.

Withdrawal of perfusate is accomplished via drainage cannulas 330a, 330b, which collect fluid from the abdominal cavity and converge into flow line 340. Outflow pump 600, in one embodiment a peristaltic suction pump, draws the perfusate from the cavity and expels it along flow line 350 toward reservoir 420. In some aspects, the pressure sensor 660 positioned along flow line 350 may monitor the suction pressure generated during drainage. Reservoir 420, supported on weight sensor 460, receives the drained perfusate. Weight sensor 460 provides continuous measurements of the combined mass of the reservoir and its contents, which is converted into real-time volume estimates of the circulating perfusate. This information allows for accurate determination of net volume changes within the abdominal cavity, and thus indirect tracking of IAV. The sensor data is further used by the controller 200 to estimate drainage efficiency in real time, enabling the detection of flow restrictions, partial occlusions, or poor outflow dynamics.

In this configuration, inflow pump 500 and outflow pump 600 operate in coordinated fashion to maintain stable circulation of perfusate between monitoring system 400 and subject 700. The balance of their respective flowrates directly determines the IAV within the cavity and thereby influences the IAP. By regulating fluid entry and withdrawal while continuously sensing delivered flowrate, the commanded flowrate of outflow pump 600, and reservoir 420 volume, system 300 enables safe and effective maintenance of a therapeutic IAV. This configuration avoids the need for intracorporeal sensors or pressure catheters, instead relying on externally mounted sensors along the main perfusion line 310 to noninvasively infer perfusion status. This approach enables fully closed-loop control of intra-abdominal perfusion using only extracorporeal instrumentation, enhancing patient safety, reducing procedural complexity, and enabling robust long-term monitoring without increasing risk of infection.

Referring now to FIG. 4, a method 1000 for automatic peritoneal perfusion control is illustrated in accordance with embodiments of the present disclosure. In various embodiments, the method 1000 may be executed by controller 200 (FIG. 1) to coordinate the operation of inflow pump 500 and outflow pump 600 during circulation of perfusate P between reservoir 420 and the peritoneal cavity of subject 700. In aspects, method 1000 may be performed by a local embedded controller, such as controller 200, or by a remote computing system, such as a cloud-based server in data communication with the perfusion system via a wired or wireless interface.

At block 1002, the controller 200 receives perfusion parameters via interface module 100. In various embodiments, the perfusion parameters are specified by an operator, such as a clinician, and include at least a predefined inflow rate (denoted as u_{1,des} and a predefined intra-abdominal volume, (denoted as “IAV,_{des}”). The inflow rate establishes the volumetric delivery rate of perfusate P through inflow pump 500 into the peritoneal cavity of subject 700. The intra-abdominal volume defines the target amount of perfusate to be retained within the cavity, which correlates directly to intra-abdominal pressure (IAP). By maintaining the IAV near a user-specified setpoint, the system balances perfusion effectiveness with avoidance of excessive intra-abdominal pressure that could otherwise lead to intra-abdominal hypertension or abdominal compartment syndrome.

A minimum acceptable drainage efficiency may be specified as a perfusion parameter θₘᵢₙ, which defines a lower bound below which outflow is considered unsafe. For example, sustained operation below θₘᵢₙ may correspond to suction-induced tissue trauma, cavitation, or occlusion of one or more drainage cannulas 330a, 330b. The interface module 100 may additionally support entry of optional parameters such as a maximum inflow rate, a maximum allowable intra-abdominal pressure, or a treatment duration, any of which may be used by controller 200 to further constrain pump operation during circulation.

Once received, the perfusion parameters are communicated to controller 200 and stored in memory 230 for use throughout method 1000. Controller 200 initializes its operating state based on the operator-specified goals and safety limits. These inputs provide the baseline targets against which subsequent sensor data, estimations, and adjustments are evaluated during execution of blocks 1004-1016, referenced below. In this way, block 1002 establishes the control objectives and safety bounds that define the subsequent behavior of the perfusion system.

At block 1004, the controller 200 initiates circulation of perfusate P between the reservoir 420 and the peritoneal cavity of subject 700 using the inflow pump 500 and the outflow pump 600. In operation, the system is primed and the at least one perfusion cannula 325 and at least one drainage cannula 330a, 330b are positioned within the peritoneal cavity of the subject 700, establishing fluid communication between the cavity and the main perfusion line 310. The inflow pump 500 propels perfusate from reservoir 420 along flow line 320 and through perfusion cannula 325 into the abdominal cavity of subject 700. Perfusate P is then withdrawn from the cavity through drainage cannulas 330a, 330b, which converge into line 340, and driven by outflow pump 600 through flow line 350 back to reservoir 420. Together, inflow pump 500 and outflow pump 600 establish a closed-loop path defined by lines 315, 320, 340, and 350, as illustrated in FIG. 3, that maintains continuous perfusion of the cavity of subject 700.

A weight sensor 460 beneath the reservoir 420 continuously measures the changing mass of the reservoir 420 to determine fluid volume. A flow-rate sensor 560 coupled along line 320 monitors the rate of perfusate delivered to subject 700 by inflow pump 500. The commanded outflow rate is obtained from the control signal sent to outflow pump 600. A pressure sensor 660 positioned along return line 350 senses the suction generated by outflow pump 600 during drainage through cannulas 330a, 330b. In some aspects, these sensors and pumps are coupled to a microcontroller or digital data acquisition circuit configured to transmit real-time data to controller 200 for analysis and feedback control (blocks 1006-1010). Collectively, the weight sensor 460, flow-rate sensor 560, and the commanded outflow rate from outflow pump 600 provide real-time information about reservoir volume, inflow rate, and outflow suction conditions during circulation.

The volumetric balance between inflow and outflow determines the intra-abdominal volume (IAV) retained within the cavity, which in turn governs intra-abdominal pressure (IAP). By coordinating operation of both pumps in conjunction with the sensor feedback, the system can increase, decrease, or stabilize the IAV in accordance with the operator-specified parameters received in block 1002. In certain embodiments, inflow pump 500 and outflow pump 600 are peristaltic pumps operated in variable-speed or reversible modes. In alternative embodiments, either or both pumps may be implemented as rotary pumps, diaphragm pumps, roller pumps, or vacuum-assisted suction devices. The system 300 may further include electronic speed controllers or feedback-regulated motor drivers configured to adjust the rotational speed of the pumps based on control signals from the controller 200. Accordingly, block 1004 establishes the dynamic circulation loop upon which subsequent steps of method 1000 are executed.

At block 1006, the reservoir volume is measured using weight sensor 460 disposed beneath reservoir 420 of FIG. 3. In an exemplary embodiment, the reservoir 420 rests directly on a load-bearing platform integrated with the weight sensor 460, which may comprise a strain-gauge, piezoelectric, or capacitive load cell configured to detect changes in mass with high resolution and temporal precision. The weight sensor 460 continuously measures and records the combined mass of the reservoir and the contained perfusate P. This measurement is converted into a volumetric value by applying the known density of the perfusate, which may be oxygenated perfluorocarbon or another biocompatible liquid. In some aspects, the conversion from mass to volume is performed automatically by controller 200 based on a lookup table or calibration curve corresponding to the specific perfusate used. In other aspects, the system may adjust the density dynamically if temperature-dependent density variations of the perfusate are known or measured. In this manner, the weight sensor 460 provides continuous real-time estimates of reservoir volume, which may be used by controller 200 to track the net amount of perfusate circulated and retained within the subject 700. Because the perfusate is nominally incompressible and not significantly absorbed or metabolized during short-duration procedures, the reservoir volume data also provides a reliable basis for estimating intra-abdominal volume (IAV) in the subject's 700 peritoneal cavity. Specifically, changes in reservoir mass correspond to net transfer of perfusate into or out of the cavity. For example, a decrease in reservoir weight indicates net fluid transfer into the cavity, and an increase reflects fluid withdrawal via drainage.

In aspects, this volume signal x₁(t) forms the primary state variable input to the controller's safety architecture. The controller 200 uses this inferred reservoir volume in blocks 1012 and 1014 to estimate drainage efficiency θ(t) and model its dynamic evolution via the state-space representation. In block 1016, the inferred volume is also used to enforce a volume-tracking term in the perfusion control policy (see, e.g., x₁=x_{1,des} in Eq. 11), thereby ensuring that IAV remains close to the operator-defined target.

The reservoir volume data obtained in block 1006 is communicated to controller 200, where it is stored and subsequently employed in drainage efficiency estimation (block 1012) and in enforcing IAV-related safety constraints during operation. In one embodiment, the weight sensor output is filtered using a moving average or low-pass filter to reduce noise and improve the stability of volume estimates, particularly during active pump transitions. This external measurement approach enables monitoring of perfusion safety without requiring additional invasive sensors within the abdominal cavity. Accordingly, the use of weight sensor 460 provides a low-complexity, high-reliability solution to infer perfusate accumulation and drainage trends, thereby enabling closed-loop control of intra-abdominal dynamics based on external sensing alone.

At block 1008, the method 1000 includes generating inflow data using flow-rate sensor 560 coupled to inflow line 320 (FIG. 3). In operation, the flow-rate sensor 560 is positioned downstream of inflow pump 500 and upstream of perfusion cannula 325 to directly monitor the volumetric flow of perfusate entering the peritoneal cavity of subject 700. The inflow data may include real-time flowrate values, instantaneous pressure fluctuations, or cumulative volume totals delivered through the inflow line 320. In embodiments where inflow pump 500 is a positive displacement device such as a peristaltic or rotary pump, the commanded flowrate u₁(t) often approximates the true delivered flow closely. However, inclusion of flow-rate sensor 560 provides independent real-time verification of delivery rate, offering redundancy and improving diagnostic confidence during perfusion. As a result, inflow data collected from flow-rate sensor 560 provides both verification of pump command accuracy and a reliable reference signal for circulation modeling.

In some aspects, the flow-rate sensor 560 may additionally monitor pulsatility, transient flow disturbances, or flow interruptions, allowing early detection of upstream occlusions, tubing kinks, or air embolism risk. The flow-rate sensor 560 may be embodied as an ultrasonic transit-time sensor, a differential-pressure transducer, a Coriolis mass flowmeter, or other conventional flow metering technology, and may additionally provide redundant measurements of inlet line pressure for diagnostic purposes.

The inflow data thus constitutes one of the three primary signals used by controller 200, establishing the true inflow rate of perfusate to the subject's peritoneal cavity and serving as the baseline against which drainage performance is later assessed. Specifically, the inflow signal u₁(t) is used in block 1012 to estimate the drainage efficiency θ(t), and in block 1014 to model efficiency dynamics via the state-space representation. In block 1016, the commanded or measured inflow rate u₁(t) is used in conjunction with the operator-specified inflow setpoint u_{1,des} to guide the perfusion safety control law and enforce volume-tracking and efficiency constraints.

At block 1010, the method 1000 includes generating outflow data, the commanded outflow rate, determined by outflow pump 600 (FIG. 3). The outflow pump 600 is fluidly coupled to drainage cannulas 330a and 330b via flow line 340 and is positioned upstream of flowline 350 leading back to reservoir 420 (FIG. 3). The controller 200 accesses the digital control signal sent to outflow pump 600, which specifies the desired, commanded outflow rate u₂ for withdrawing perfusate P from the peritoneal cavity of subject 700. The commanded outflow rate provides a direct representation of the pump's intended performance and is continuously tracked by the controller 200. During operation, the controller 200 compares the commanded outflow rate u₂ with the measured inflow rate u₁(t) (block 1008) and the rate of change of reservoir volume \dot{x}₁ (block 1006) to infer the true drainage rate. This inference supports calculation of the drainage efficiency parameter θ, which reflects how closely the actual drainage matches the commanded rate (see block 1012). Together with the inflow and reservoir measurements, the outflow data completes the triad of primary inputs that enable controller 200 to regulate intra-abdominal volume (IAV), intra-abdominal pressure (IAP), and drainage efficiency in real time.

In certain aspects, the pressure sensor 660 positioned proximate to outflow pump 600 may measure suction pressure of outflow pump 600 as perfusate P is withdrawn from the peritoneal cavity via drainage cannulas 330a, 330b. In this aspect, the suction pressure measured by pressure sensor 660 provides real-time data indicative of conditions within the return line 350 during drainage. The pressure sensor 660 captures the negative pressure required to withdraw perfusate P from the peritoneal cavity via drainage cannulas 330a, 330b. Elevated suction pressures may reflect increased resistance in the outflow path, which can arise from partial occlusion of the cannulas, tissue apposition, or cavitation at the pump inlet. In some aspects, pressure sensor 660 may be embodied as a MEMS-based gauge or differential transducer, a piezoresistive element, or a strain-based diaphragm sensor, and may be configured to compensate for pulsatile pressure artifacts produced by peristaltic actuation.

In an aspect, the combination of reservoir 420 perfusate P volume data (block 1006), inflow data (block 1008), and outflow data (block 1010) provides sufficient information for controller 200 to regulate perfusion without requiring any additional sensors to be placed inside the peritoneal cavity. As illustrated in FIG. 3, the weight sensor 460 is positioned beneath the reservoir 420 and is configured to measure the mass of the contained perfusate P, which is converted into real-time volume estimates used to infer intra-abdominal volume (IAV) and net fluid accumulation in the subject 700. The flow-rate sensor 560 is positioned along the inflow line 320 downstream of inflow pump 500 and upstream of perfusion cannula 325 and is configured to directly measure the volumetric flowrate of perfusate entering the peritoneal cavity. This data provides verification of commanded inflow performance and serves as a baseline for modeling circulation dynamics in later steps (e.g., blocks 1012-1014). The outflow pump 600 provides a commanded outflow rate that is tracked by the controller 200 during withdrawal of perfusate P. In certain aspects, the suction pressure measured by the pressure sensor reflects hydraulic resistance in the outflow path and is used by the controller 200 to detect occlusion, tissue apposition, or cavitation-related disturbances in the drainage path.

The weight sensor 460, flow-rate sensor 560, and outflow rates from outflow pump 600, enable the system 10 to infer key internal physiological variables such as intra-abdominal volume (IAV), intra-abdominal pressure (IAP), and drainage efficiency θ using only external, non-invasive instrumentation. The outputs from these sensors are fed to controller 200, which utilizes this data in subsequent method blocks to estimate true drainage flow (block 1012), identify efficiency dynamics (block 1014), and apply perfusion safety control (block 1016). By relying on externally positioned sensors, the system minimizes invasiveness, reduces the number of cannulations, and avoids the risks associated with direct intra-abdominal pressure measurement. This externalized sensing architecture therefore achieves real-time monitoring and regulation while maintaining a simplified and safer experimental or clinical setup.

At block 1012, the method 1000 proceeds by estimating a drainage efficiency parameter θ that characterizes the effectiveness of fluid removal from subject 700. In one embodiment, drainage efficiency θ is defined as the ratio between the true outflow rate from the peritoneal cavity, u_{2,true}(t), and the commanded outflow rate u₂(t) sent to outflow pump 600. Thus, the drainage efficiency θ can be determined by the following equation:

$$θ = \frac{{u}_{2 , true}}{{u}_{2}} \qquad ( Eqn . 1 )$$

where u_{2,true} is the actual fluid outflow rate (L/s), and u₂ is the commanded outflow rate of outflow pump 600 (L/s). A value of θ=1 indicates that outflow pump 600 is removing fluid at the commanded rate. In another aspect, a value of θ significantly less than 1 indicate partial occlusion, cavitation, or other drainage impairment.

In aspect, the present disclosure utilized an indirect estimation strategy based on the monitored reservoir volume change to measure u_{2,true}. Specifically, x₁(t) corresponds to the perfusate (PFC) volume in reservoir 420 (L), u₁(t) corresponds to the commanded inflow rate of inflow pump 500 (L/s), and u₂ is the commanded outflow rate of outflow pump 600 (L/s). The commanded inflow rate of inflow pump 500 being u₁(t) leads to the following state equation for the volume of perfusate P in reservoir 420:

$${\dot{x}}_{1} = θ {u}_{2} - {u}_{1} \qquad ( Eqn . 2 )$$

where \dot{x}₁(t) is the time derivative of the volume of PFC in the reservoir 420. In an aspect, \dot{x}₁(t) is obtained from the weight sensor 460 disposed beneath reservoir 420, which continuously measures reservoir weight and converts it to volume. The assumption of fluid incompressibility ensures that inflow from inflow pump 500 is accurately delivered as commanded, allowing estimation of the true drainage rate from discrepancies between measured reservoir volume dynamics and commanded pump signals of outflow pump 600.

The controller 200 executes a regression-based estimator to solve for θ. In one embodiment, the estimator is implemented as a continuous-time least squares problem with exponential forgetting:

$$\frac{min}{θ} {\int }_{∞}^{t} ( {( {\dot{x}}_{1} ( τ ) + {u}_{1} ( τ ) - θ ( τ ) {u}_{2} ( τ ) )}^{2} {e}^{- λ ( t - τ )} d τ \qquad ( Eqn . 3 )$$

where λ is a forgetting factor (0.02 s⁻¹ in an aspect) corresponding to a memory horizon of approximately 50 seconds. This formulation emphasizes recent data, allowing the estimate to adapt to dynamic changes in drainage efficiency over time. The analytic solution to the regression of Eqn. 3 is:

$$θ ( t ) = {\int }_{∞}^{t} \frac{{\int }_{- ∞}^{t} ( {\dot{x}}_{1} ( τ ) + {u}_{1} ( τ ) ) {u}_{2} ( τ ) {e}^{- λ ( t - τ )} d τ}{{\int }_{- ∞}^{t} {u}_{2}^{2} ( τ ) {e}^{- λ ( t - τ )} d τ} \qquad ( Eqn . 4 )$$

In an aspect, the regression of Eqn. 4 may be implemented digitally, for example using Simulink with a 0.01-second integration step. Initial conditions for the numerator and denominator integrals can be chosen such that θ(0) reflects an assumed baseline efficiency, e.g., 95%. The estimator output θ(t) thus provides a real-time indication of drainage performance without the need for invasive flowrate sensors.

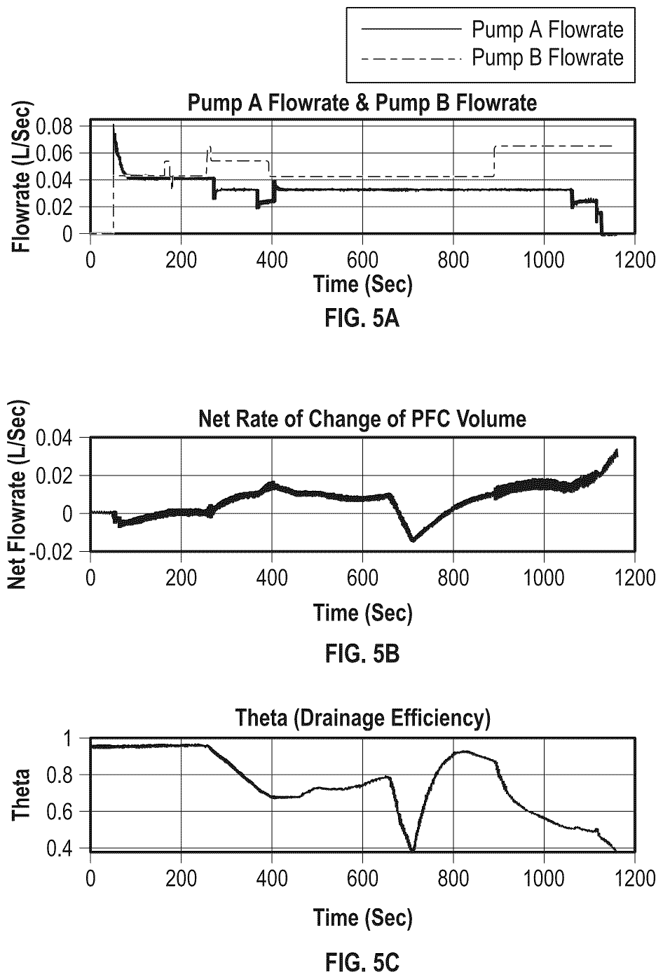

The controller 200 therefore produces a continuous real-time estimate of drainage efficiency θ(t) of system 10. The controller receives as inputs the commanded inflow rate u₁(t) provided to inflow pump 500, the commanded outflow rate u₂(t) provided to outflow pump 600, and the rate of change of reservoir volume \dot{x}₁(t) derived from weight sensor 460 beneath reservoir 420. The controller 200 applies the least-squares regression of Eqn. (3) with exponential forgetting to continuously update an estimate of drainage efficiency θ(t) The controller 200 then outputs monitoring data that includes the commanded pump flow rates u₁(t) and u₂(t) (FIG. 5A), the net reservoir volume change \dot{x}₁(t) (FIG. 5B), and the resulting efficiency estimate θ(t) (FIG. 5C). As illustrated in FIGS. 5A-5C, FIG. 5A represents the commanded pump flow rates that establish the intended perfusion dynamics, FIG. 5B reflects the actual fluid balance within reservoir 420, and FIG. 5C provides a safety metric indicating the effectiveness of fluid drainage.

At block 1014, the controller 200 applies a data-driven modeling routine to represent the evolution of drainage efficiency over time. Building on the real-time estimator of block 1012, the controller 200 defines an occlusion-related state variable x₂(t) that governs the changes in efficiency. In one embodiment, the state variable evolves according to a linear differential equation:

$${\dot{x}}_{2} = {α}_{1} {u}_{1} + {α}_{2} {u}_{2} + {α}_{3} {x}_{2} + {α}_{4} , θ ( t ) = \frac{{e}^{{x}_{2}}}{1 + {e}^{{x}_{2}}} \qquad ( Eqn . 5 )$$

where x₂ is the drainage efficiency and θ is related to the data-driven state variable x₂ through a sigmoidal function that bounds θ between 0 and 1. This bounded representation ensures that the model output always remains physically interpretable, with θ≈1 indicating unobstructed drainage and θ≈0 corresponding to near-total occlusion. The dynamics of the occlusion-related state variable, x₂, are governed by a simple linear data-driven state equation with unknown constant parameters α₁, α₂, α₃, and α₄. Furthermore, in an aspect of the present disclosure, u₁ is the inflow rate of inflow pump 500 and u₂ is the commanded outflow rate to outflow pump 600.

The controller 200 determines parameter values by fitting Eqn. (5) to experimental datasets, for example using parameter estimation routine to minimize the squared error between measured drainage efficiency curves and the predicted θ(t). In one embodiment, identification yielded parameter values of α₁=0.5870, α₂=−1.6581, α₃=−0.0248, and α₄=0.0900. The signs and magnitudes of these values yield physically consistent behavior. For example, the positive value of α₁ indicates that increases in the commanded inflow rate u₁(t) tend to improve drainage efficiency. Physiologically, this corresponds to perfusate P entering the cavity under pressure, which can both elevate intra-abdominal pressure to facilitate clearance and physically displace bowel surfaces or omental tissue that might otherwise obstruct drainage cannulas 330a and 330b. In contrast, the negative value of α₂ reflects that aggressive suction commands to outflow pump 600 tend to reduce efficiency by drawing tissue into the drainage cannulas 330a and 330b, inducing partial occlusion, or promoting cavitation in the outflow line. The parameter α₃ is also negative, ensuring that the system 10 remains stable over time, such that deviations in the efficiency state variable x₂, decay rather than diverge. Finally, the offset parameter 4 defines the baseline tendency of the system 10, such that when both inflow and outflow commands are zero, the predicted steady-state efficiency θ is close to unity (0.9743 in one embodiment). As a result, the full state-space drainage efficiency model becomes:

$${\dot{x}}_{1} = θ {u}_{2} - {u}_{1} , {\dot{x}}_{2} = {α}_{1} {u}_{1} + {α}_{2} {u}_{2} + {α}_{3} {x}_{2} + {α}_{4} \qquad ( Eqn . 6 )$$

where, x₁ is the perfusate P volume in the reservoir 420, x₂ is drainage efficiency, and

$$θ = \frac{{e}^{{x}_{2}}}{1 + {e}^{{x}_{2}}} .$$

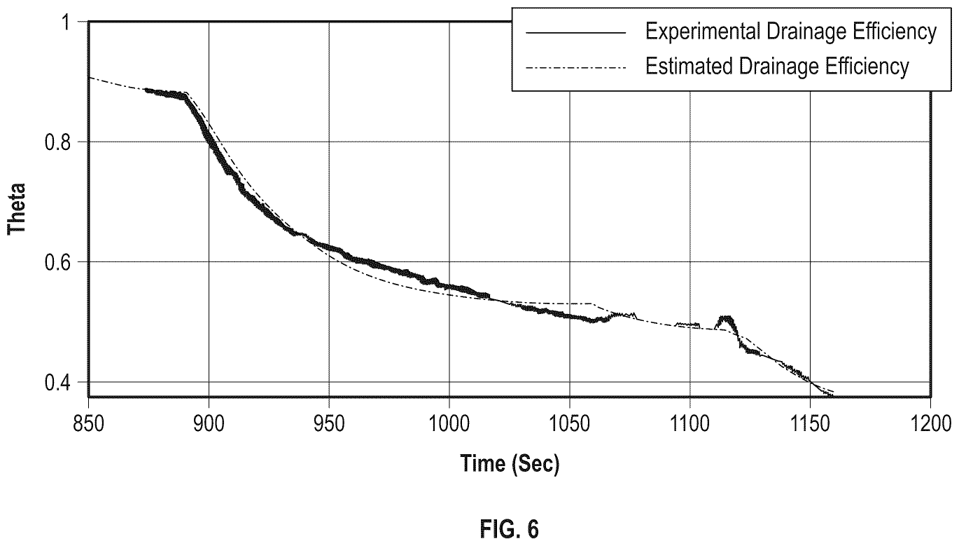

The outputs of this modeling process are illustrated in FIG. 6, which compares the experimental drainage efficiency θ(t) to the modeled θ(t) over time. In an aspect, the close alignment between the experimental curve and modeled curve confirms that the state-space model accurately reproduces observed dynamics while remaining computationally tractable for real-time implementation. The graph demonstrates that the controller's 200 state-space model closely tracks observed efficiency trends, providing a reliable and computationally simple representation of drainage dynamics.

Accordingly, at block 1014 the controller 200 transforms the raw estimator output of block 1012 into a predictive drainage efficiency state-space model. This model allows the controller 200 to simulate efficiency under different pump commands and anticipate unsafe trends. Accordingly, in this aspect the identified state-space drainage efficiency model generated at block 1014 supplies the predictive framework that the controller 200 subsequently employs in block 1016 to distinguish safe from unsafe drainage conditions and to enforce barrier-based safety control.

At block 1016, the method 1000 applies a perfusion safety control routine to determine and implement adjustments to circulation of perfusate between inflow pump 500 and outflow pump 600. At block 1016, the controller 200 employs the state variable x₂ determined in the state-space model from Eqn. 5 from block 1014, to ensure that occlusion is prevented during perfusion by maintaining the desired fluid volume in the reservoir 420, tracking the user-specified perfusion flowrate, and insuring that

$$θ = \frac{{e}^{{x}_{2}}}{1 + {e}^{{x}_{2}}}$$

remaining above the safety threshold. In order to ensure this, the controller 220 employs a safety barrier function, defined as:

$${f}_{b} ( {x}_{2} , {x}_{2 , min} ) = {x}_{2} - {x}_{2 , min} \qquad ( Eqn . 7 )$$

where x_{2,min} is a safe lower bound on x₂. The monotonic/sigmoidal relationship between x₂ and drainage efficiency θ implies that

$${x}_{2 , min} = ln \frac{{θ}_{min}}{1 - {θ}_{min}}$$

for any desired minimum drainage efficiency, θₘᵢₙ. In an aspect, a positive value of the barrier function ƒ_{b} indicates that drainage efficiency is within safe bounds, while a negative value indicates unsafe conditions or impending occlusion. Moreover, ƒ_{b} should recover to a positive value if it starts from a negative initial condition. To ensure recovery from unsafe conditions, the time derivative of the safety barrier function is constrained according to:

$$\frac{{df}_{b}}{df} \geq - {λ}_{2} {f}_{b} \qquad ( Eqn . 8 )$$

where λ₂ is a constant dictating the rate of recovery of the barrier function ƒ_{b}. In an aspect, Eqn. 6 is substituted into Eqn. 7, resulting in the following constraint:

$${\dot{x}}_{2} \geq - {λ}_{2} ( {x}_{2} - {x}_{2 , min} ) = {λ}_{2} ( {x}_{2} - {x}_{2 , min} ) \qquad ( Eqn . 9 )$$

In another aspect, the \dot{x}₂ from the state space model of Eqn. 6 into Eqn. 8, results in the following safety constraint:

$${α}_{1} {u}_{1} + {α}_{2} {u}_{2} + {α}_{3} {x}_{2} + {α}_{4} \geq {λ}_{2} ( {x}_{2} - {x}_{2 , min} ) \qquad ( Eqn . 10 )$$

Furthermore, at block 1016, the controller 200 employs a real-time control model continuously during the operation of system 10. This control model allows the perfusion safety control to track the user-specified flowrate, u_{1,des} and the user-specified volume of PFC in the reservoir 420. The controller 200 solves this control model continuously during system 10 operation by applying a barrier-constrained control formulation. The mathematical formulation solved by the controller 200 in real-time is the following:

$$\begin{matrix}min \\ {u}_{1} , {u}_{2}\end{matrix} {( {u}_{1} - {u}_{1 , des} )}^{2} sub . to : \qquad ( Eqn . 11 )$$

$${α}_{1} {u}_{1} + {α}_{2} {u}_{2} + {α}_{3} {x}_{2} + {α}_{4} \geq {λ}_{2} ( {x}_{2} - {x}_{2 , min} )$$

$${\dot{x}}_{1} = \frac{{e}^{{x}_{2}}}{1 + {e}^{{x}_{2}}} {u}_{2} - {u}_{1} = {λ}_{1} ( {x}_{1 , des} - {x}_{1} )$$

$${\dot{x}}_{2} = {α}_{1} {u}_{1} + {α}_{2} {u}_{2} + {α}_{3} {x}_{2} + {α}_{4}$$

The controller 200 is configured to solve the real-time control model with the goal of minimizing the difference between the user-specified ideal perfusion flowrate, u_{1,des}, and the true pump 500 flowrate, u₁. Achieving the ideal flowrate exactly is not always feasible, given the potential need for ensuring safety through flowrate curtailment. Accordingly, in an aspect the control model is performed subject to the safety barrier function as an inequality constraint. This allows the controller 200 to assess perfusion safety, such that if the drainage efficiency θ is initially safe, it will subsequently remain the same, and therefore safe.

In the perfusion safety control, the controller 200 then causes system 10 to determine whether the safety barrier function barrier is a positive number and therefore is safe with no occulation. At every instant in time, the controller 200 assumes that u₁=u_{1,des} and then to obtains u₂ flowrate of pump 600 through the following inequality constraint:

$${\dot{x}}_{1} = \frac{{e}^{{x}_{2}}}{1 + {e}^{{x}_{2}}} {u}_{2} - {u}_{1 , des} = {λ}_{1} ( {x}_{1 , des} - {x}_{1} ) \qquad ( Eqn . 12 )$$

$$⇒ {u}_{2} = \frac{{λ}_{1} ( {x}_{1 , des} - {x}_{1} ) + {u}_{1 , des}}{\frac{{e}^{{x}_{2}}}{1 + {e}^{{x}_{2}}}}$$

Once u₂ has been determined, the controller 200 causes the perfusion safety control to determine if the resulting unconstrained control policy satisfies the barrier constraint in Eqn. 10. If the constraint is satisfied, the unconstrained values of u₁ and u₂ are implemented. When the constraint is satisfied, the controller 200 does not need to control the flowrate of pump 500, u₁. Rather, the controller 200 may only control the flowrate of pump 600, u₂ to ensure the desired PFC volume is in the reservoir 420 during operation.

However, if the resulting u₁ and u₂ values of the unconstrained control policy violate the safety barrier constraint of Eqn. 10, the controller 200 then causes the system 10 to compute the constrained values of u₁ and u₂ using the below formula:

$$[ \begin{matrix}- 1 & θ \\ {α}_{1} & {α}_{2}\end{matrix} ] [ \begin{matrix}{u}_{1} \\ {u}_{2}\end{matrix} ] = [ \begin{matrix}{λ}_{1} ( {x}_{1 , des} - {x}_{1} ) \\ {λ}_{2} ( {x}_{2 , min} - {x}_{2} ) - {α}_{3} {x}_{2} - {α}_{4}\end{matrix} ] \qquad ( Eqn . 13 )$$

Once the controller 200 has caused the system to determine the constrained values of u₁ and u₂ by Eqn. 13, the controller 200 commands the constrained u₁ value to pump 500 and the u₂ to pump 600. In accordance with aspects, the controller 200 controls the flow rate of both pumps 500 and 600 to improve the drainage efficiency θ while achieving desired perfusate P volume in the reservoir 420.

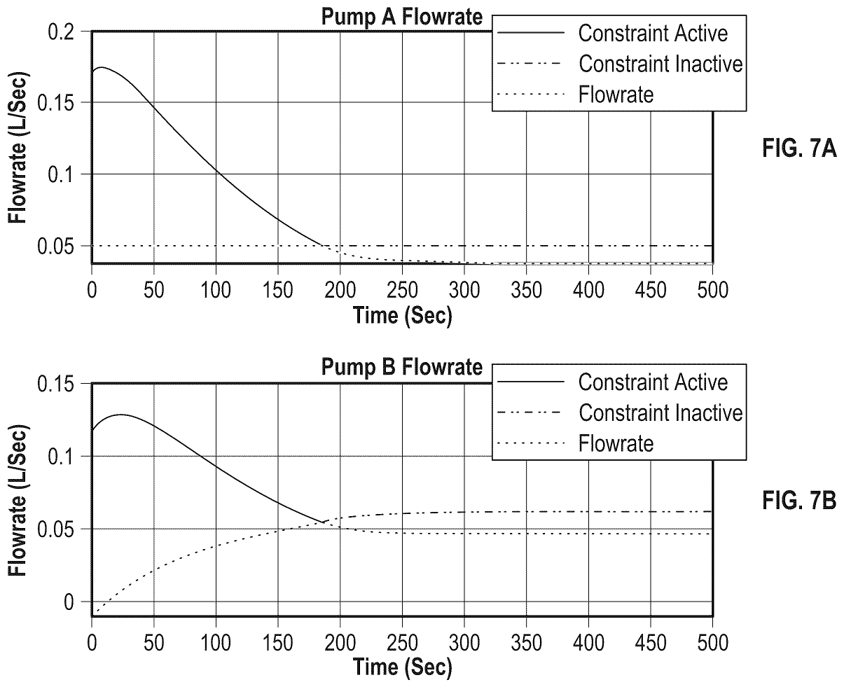

Accordingly, at block 1016 the controller 200 implements a switching control policy. During normal operation, unconstrained pump commands track the operator-defined perfusion parameters. When unsafe drainage conditions are detected, the constrained policy overrides the commands to enforce efficiency recovery. This ensures that perfusion remains effective and safe under both normal and occlusion-prone conditions, as illustrated in FIGS. 7A and 7B.

In operation, execution of method 1000 proceeds sequentially through blocks 1002-1016 to achieve automated and safe peritoneal perfusion. At block 1002, controller 200 receives operator-defined perfusion parameters including a target intra-abdominal volume, desired inflow rate, and minimum acceptable drainage efficiency. At block 1004, circulation of perfusate is initiated between reservoir 420 and subject 700 using inflow pump 500 and outflow pump 600. At blocks 1006-1010, monitoring data is collected, including reservoir volume from weight sensor 460, inflow data from flow-rate sensor 560, and outflow suction pressure from pressure sensor 660. At block 1012, controller 200 generates an estimate of drainage efficiency θ(t) using least-squares regression with exponential forgetting, and at block 1014, controller 200 identifies a state-space model of efficiency dynamics, defining occlusion-related state variable x₂(t). At block 1016, controller 200 applies a perfusion safety control routine that evaluates the barrier function and applies a perfusion safety control routine to determine and implement adjustments to circulation of perfusate between inflow pump 500 and outflow pump 600. The controller 200 employs the state variable x₂ determined in the state-space model of Eqn. (5) from block 1014 to ensure that occlusion is prevented during perfusion by maintaining the desired fluid volume in reservoir 420, tracking the user-specified perfusion flowrate, and ensuring that drainage efficiency θ=x₂ remains above a safety threshold. To enforce this condition, the controller 200 applies a safety barrier function as defined in Eqn. (7) and its associated constraints (Eqns. (8)-(10)). At every instant in time, the controller assumes that u₁=u₁,des, employs Eqn. (11) to solve for u₂, and then checks whether the resulting unconstrained control policy satisfies the safety barrier constraint of Eqn. (9). If the safety barrier constraint is satisfied, the unconstrained values of u₁ and u₂ are implemented. However, if the unconstrained values of u₁ and u₂ would violate the safety barrier constraint, then the controller applies Eqn. (12) to compute the constrained values of u₁ and u₂, and commands these constrained values to inflow pump 500 and outflow pump 600, respectively. In this way, the controller 200 implements a switching control policy that allows normal operator-specified perfusion commands to proceed under safe conditions, while automatically substituting constrained pump commands during unsafe or occlusion-prone conditions.

To support the development and validation of the drainage efficiency estimation algorithm, the state-space model of occlusion dynamics, and the safety-constrained control logic described in blocks 1012 through 1016 of method 1000, an in vivo experimental embodiment was conducted. The experiment served to demonstrate the feasibility of noninvasive sensor-based estimation and control using the disclosed system. In this exemplary embodiment, the perfusion flow pump system 300 was implemented in an in vivo study to validate drainage efficiency estimation and occlusion detection. The subject 700 was an anesthetized laboratory animal in which a perfusion cannula 320 and two drainage cannulas 330a, 330b were inserted into the peritoneal cavity. The inflow pump 500 delivered oxygenated perfluorocarbon (PFC) from the reservoir 420 to the cavity, while the outflow pump 600 withdrew fluid via the drainage cannulas. The reservoir 420 was positioned on a load cell 460 to measure fluid volume changes, and a flowrate sensor 560 monitored inflow conditions. The experiment included 1160 seconds of circulation. At approximately 440 seconds, one of the drainage cannulas was manually occluded, and at approximately 660 seconds, both cannulas were simultaneously occluded. These events reduced true drainage relative to commanded drainage, lowering drainage efficiency. The estimator algorithm, executed by the controller 200, successfully detected these occlusion events by comparing load cell data with pump command histories. The experimental results confirmed the utility of drainage efficiency as an occlusion metric. Data from this experiment was further used to identify parameters for a dynamic state-space model of drainage efficiency and to demonstrate the performance of a safety-constrained controller. While this embodiment describes a laboratory animal study, the same configuration may be adapted for human subjects or other perfusion contexts without departing from the scope of the present disclosure.

FIGS. 5A-5C illustrate representative experimental results of the drainage efficiency estimation described in connection with block 1012. FIG. 5A depicts the commanded inflow and outflow rates, u₁(t) for inflow pump 500 and u₂(t) for outflow pump 600. FIG. 5B shows the net rate of change of reservoir volume, \dot{x}₁(t), as measured by weight sensor 460 disposed beneath reservoir 420 and converted to volumetric flow (L/s). FIG. 5C presents the estimated drainage efficiency, θ(t), computed in real time using the regression-based estimator of Eqns. (2)-(4).

The data in FIGS. 5A-5C can be divided into five characteristic phases. In the first phase (0-250 seconds), the commanded inflow and outflow rates are balanced, resulting in near-zero \dot{x}₁(t) and an efficiency θ≈1, indicating unobstructed drainage. In the second phase (approximately 250-400 seconds), the outflow command u₂(t) exceeds inflow command u₁(t) in order to achieve net drainage. During this period, \dot{x}₁(t) becomes positive as reservoir volume increases, while θ decreases, reflecting that true drainage is less than commanded due to rising suction resistance. In the third phase (400-660 seconds), pump commands are moderated to achieve gradual drainage. The reservoir rate of change remains positive, and θ recovers partially, though disturbances such as pinching one of the two drainage cannulas produce only minor transient effects since the second cannula remains patent. In the fourth phase, beginning at approximately 660 seconds, both drainage cannulas 330a, 330b are deliberately occluded. The estimator immediately reports a sharp and sustained drop in θ(t), consistent with severe drainage impairment. This transient demonstrates the sensitivity of the estimator to full outflow occlusion. In the fifth phase (after ˜800 seconds), aggressive suction commands are applied to outflow pump 600. The reservoir volume increases more rapidly, but θ again declines, evidencing the onset of cavitation and the diminished effectiveness of drainage under excessive suction.

Collectively, FIGS. 5A-5C demonstrate two important capabilities of the estimator. First, the estimator reliably detects total outflow occlusion, as shown by the precipitous decline in θ during phase four. Second, the estimator captures the reduced efficiency that occurs whenever outflow command u₂(t) substantially exceeds inflow command u₁(t), a condition associated with cavitation or suction-induced tissue trauma. These observations align with contemporaneous visual confirmation during the animal experiments and underscore the value of θ(t) as a safety-critical variable for real-time perfusion monitoring.

FIG. 6 illustrates the comparison between experimental drainage efficiency and estimated drainage efficiency as a function of time. The experimental efficiency is derived from direct fluid balance observations during animal testing, whereas the estimated efficiency is produced by the regression-based estimator of block 1012. The close alignment between the two curves demonstrates that the estimator faithfully reproduces measured efficiency trends without requiring invasive flow sensors. In particular, both traces show a gradual decline in efficiency from approximately 0.9 to 0.4 over the 850-1150 second interval, capturing the effect of progressive drainage impairment. Small deviations between the experimental and estimated curves are attributable to transient disturbances such as catheter pinching, yet the estimator remains stable and tracks the long-term trend. These results validate the modeling approach by confirming that efficiency can be inferred from reservoir dynamics alone, thereby enabling real-time feedback in a clinical setting.

FIGS. 7A and 7B depict commanded flowrates for inflow pump 500 and outflow pump 600, respectively, under conditions with the perfusion safety constraint inactive versus active. In both graphs, the flowrates when the safety barrier constraint is enforced, an unconstrained operation, and an actual applied flowrate are represented. In FIG. 7A, inflow pump 500 follows the operator-specified command closely under unconstrained conditions, but when the barrier becomes active, the commanded inflow is curtailed to prevent excessive intra-abdominal pressure. In FIG. 7B, the outflow pump 600 demonstrates a complementary adjustment: when the safety constraint is triggered, suction is reduced relative to the unconstrained case, thereby preventing efficiency collapse due to occlusion. Together, FIGS. 7A and 7B illustrate the switching policy implemented at block 1016, wherein unconstrained control values are implemented under safe conditions, but constrained values are imposed when θ(t) falls below the minimum safety threshold.

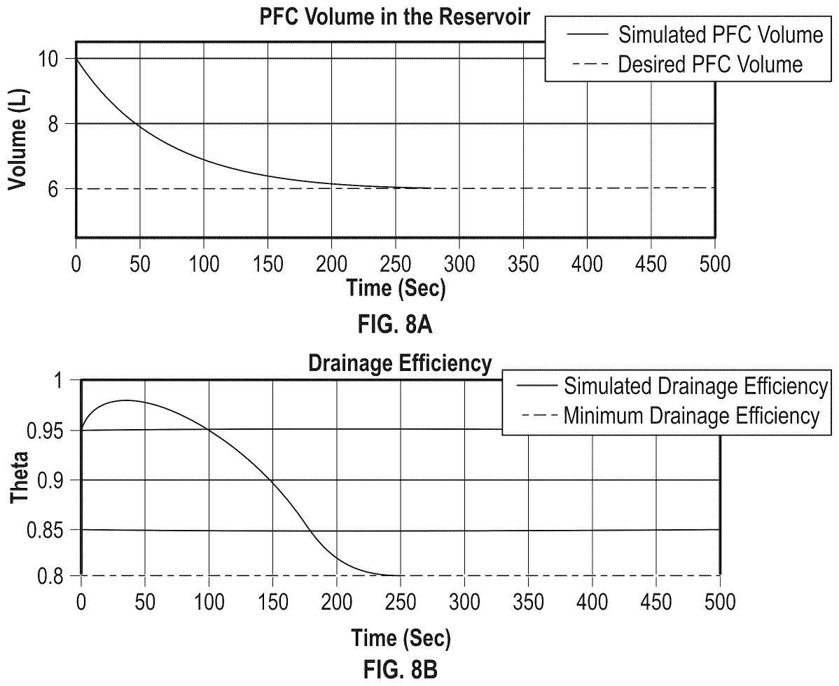

FIGS. 8A and 8B illustrate simulated perfusion dynamics under the safety-constrained controller. FIG. 8A plots the simulated PFC volume in reservoir 420 against the operator-specified desired volume. The simulated volume converges smoothly to the target, demonstrating that the controller is able to achieve user-defined IAV despite perturbations. FIG. 8B plots the corresponding drainage efficiency θ(t). The simulation confirms that θ(t) remains above the minimum acceptable threshold throughout operation, even when flowrates are reduced to enforce barrier constraints. This figure emphasizes that the safety barrier function is effective in preserving drainage efficiency while still meeting the clinical objective of achieving the target perfusate volume.

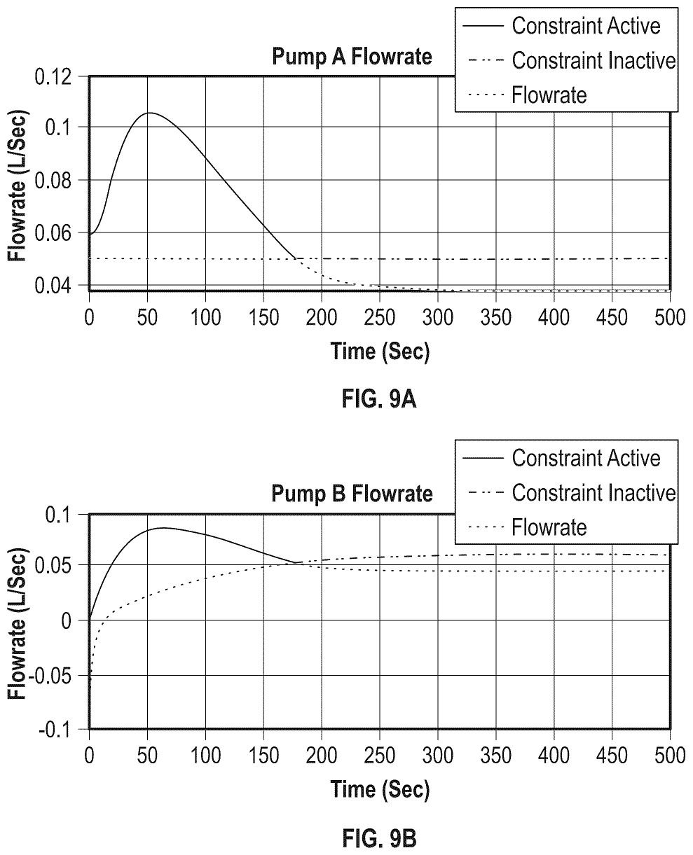

FIGS. 9A and 9B present simulated flowrates of inflow pump 500 and outflow pump 600, respectively, under scenarios with the safety constraint inactive versus active. In both figures, the unconstrained command, the constrained command under active safety enforcement, and the applied flowrate, are represented in FIG. 9A, the unconstrained inflow rate initially overshoots, whereas the constrained command damps the response to avoid unsafe intra-abdominal pressure buildup. In FIG. 9B, the outflow rate is similarly reduced by the constrained controller to prevent suction-induced occlusion. The differences between the constraint active and inactive line represent the adaptive action of the safety barrier function, which actively modifies both inflow and outflow pump commands to maintain safe and effective perfusion.

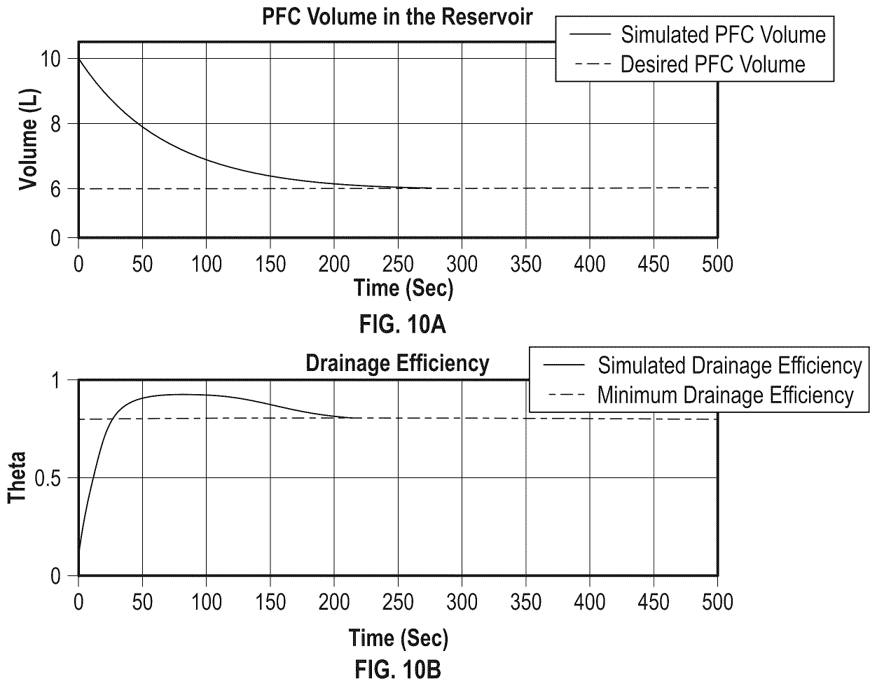

FIGS. 10A and 10B depict additional simulation results demonstrating the interaction between perfusate volume regulation and drainage efficiency maintenance. FIG. 10A shows that the simulated PFC volume converges steadily to the operator-defined target volume, confirming the system's ability to meet clinical input requirements. FIG. 10B shows the drainage efficiency θ(t), which stabilizes near unity before declining slightly but remaining consistently above the predefined minimum threshold. These plots collectively highlight the dual-objective nature of the perfusion safety controller: achieving desired perfusate volume while simultaneously preserving drainage efficiency above a safety margin.

In an aspect of the present disclosure, the automatically controlled peritoneal perfusion system 10 may be implemented as or incorporated within a hyperthermic intraperitoneal chemotherapy (HIPEC) system, a specific type of peritoneal perfusion system. In this configuration, the system delivers a heated chemotherapeutic perfusate to the peritoneal cavity of a subject, circulates the fluid through the cavity, and maintains the desired intra-abdominal volume (IAV), pressure (IAP), and safety parameters throughout the HIPEC procedure. The perfusate may include a solution of one or more cytotoxic drugs dissolved in a physiologically compatible carrier fluid and heated to a therapeutic temperature. In this way, the automatically controlled peritoneal perfusion system 10 of the present disclosure enables real-time, closed-loop control of perfusion safety and efficacy in both HIPEC applications and non-chemotherapeutic procedures such as peritoneal dialysis or peritoneal oxygenation.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different example embodiments provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A method for automatically controlled peritoneal perfusion, comprising:
receiving perfusion parameters, the perfusion parameters including at least a predefined intra-abdominal volume and a predefined inflow rate;
circulating a perfusate between a reservoir and a cavity of a subject using an inflow pump and an outflow pump;
measuring a reservoir volume using a weight sensor positioned beneath the reservoir;
generating inflow data using a flow-rate sensor coupled to the inflow pump;
accessing a commanded outflow rate from the outflow pump;
estimating a drainage efficiency of the outflow pump based on at least the reservoir volume, the inflow rate, and the commanded outflow rate;
identifying a drainage efficiency model from experimental or operational data to represent efficiency dynamics; and
applying a perfusion safety control routine that evaluates a safety barrier function based on the drainage efficiency model and adjusting circulation of perfusate between the inflow pump and the outflow pump based on the evaluated safety barrier function.

2. The method of claim 1, wherein the perfusion parameters further include a minimum drainage efficiency threshold.

3. The method of claim 1, wherein estimating the drainage efficiency comprises solving a least-squares regression with exponential forgetting to adaptively update the efficiency estimate over time.

4. The method of claim 1, wherein identifying the drainage efficiency model comprises fitting a state-space representation of drainage efficiency dynamics to experimental or operational data.

5. The method of claim 4, wherein the state-space model includes an occlusion-related state variable governed by a linear differential equation with constant parameters.

6. The method of claim 1, wherein applying the perfusion safety control routine comprises evaluating a safety barrier function that maintains drainage efficiency above the minimum drainage efficiency threshold.

7. The method of claim 1, wherein applying the perfusion safety control routine comprises:
assuming the inflow pump rate equals a desired inflow rate;
computing an unconstrained outflow pump command;
determining whether the unconstrained pump commands satisfy the safety barrier function; and
computing constrained pump commands that maintain safety when the unconstrained pump commands violate the safety barrier function.

8. The method of claim 1, wherein the reservoir volume is measured by the weight cell disposed beneath the reservoir.

9. The method of claim 1, wherein the inflow data is measured by the flow-rate sensor coupled to the inflow pump.

10. The method of claim 1, wherein the outflow data is measured by the pressure sensor disposed along a return line of the outflow pump.

11. The method of claim 1, wherein the peritoneal perfusion is a hyperthermic intraperitoneal chemotherapy (HIPEC) procedure and the method is performed during the HIPEC procedure.

12. An automatically controlled peritoneal perfusion system, comprising:
a reservoir configured to hold a perfusate;
an inflow pump fluidly coupled to the reservoir and configured to deliver the perfusate to a peritoneal cavity of a subject;
an outflow pump fluidly coupled to the peritoneal cavity and configured to withdraw the perfusate and return the perfusate to the reservoir;
a plurality of sensors comprising at least one weight sensor, at least one flow-rate sensor, and at least one pressure sensor;
at least one processor; and
at least one memory storing instructions, which when executed by the processor, cause the system to:
receive perfusion parameters including at least a target intra-abdominal volume and a target inflow rate;
circulate the perfusate between the reservoir and the peritoneal cavity using the inflow pump and the outflow pump;
measure a reservoir volume using the weight sensor;
generate inflow data using the flow-rate sensor;
access commanded outflow rate from the outflow pump;
estimate a drainage efficiency of the outflow pump based at least on the reservoir volume, the inflow data, and the commanded outflow rate; and
apply a perfusion safety control routine that evaluates the estimated drainage efficiency relative to the perfusion parameters and adjusts operation of the inflow pump and the outflow pump based on the evaluation.

13. The system of claim 12, wherein the processor is further configured to receive perfusion parameters that include a minimum drainage efficiency threshold.

14. The system of claim 12, wherein the instructions, when executed by the processor, further cause the system to:
estimate drainage efficiency by solving a least-squares regression with exponential forgetting to adaptively update the efficiency estimate over time.

15. The system of claim 12, wherein the instructions, when executed by the processor, further cause the system to:
identify a drainage efficiency model by fitting a state-space representation of drainage efficiency dynamics to experimental or operational data.

16. The system of claim 15, wherein the state-space model includes an occlusion-related state variable governed by a linear differential equation with constant parameters.

17. The system of claim 12, wherein the instructions, when executed by the processor, further cause the system to evaluate a safety barrier function that maintains drainage efficiency above the minimum drainage efficiency threshold.

18. The system of claim 12, wherein the instructions, when executed by the processor, further cause the system to assume an inflow pump rate equal to a desired inflow rate;
compute an unconstrained outflow pump command;
determine whether the unconstrained pump commands satisfy the safety barrier function; and
when the unconstrained pump commands violate the safety barrier function, compute constrained pump commands that maintain safety.

19. The system of claim 12, wherein the weight sensor is a load cell disposed beneath the reservoir to measure reservoir volume.

20. The system of claim 12, wherein the flow-rate sensor is coupled to the inflow pump to measure inflow data.

21. The system of claim 12, wherein the pressure sensor is disposed along a return line of the outflow pump to measure outflow data.

22. The system of claim 12, wherein the peritoneal perfusion system is a hyperthermic intraperitoneal chemotherapy (HIPEC) system and the peritoneal perfusion system is configured for use during a HIPEC procedure.

23. A non-transitory computer-readable medium storing instructions that, when executed by at least one processor, cause a system for automated peritoneal perfusion to:
receive perfusion parameters, the perfusion parameters including at least a predefined intra-abdominal volume and a predefined inflow rate;
circulate a perfusate between a reservoir and a cavity of a subject using an inflow pump and an outflow pump;
measure a reservoir volume using a weight sensor positioned beneath the reservoir;
generate inflow data using a flow-rate sensor coupled to the inflow pump;
access a commanded outflow rate from the outflow pump;
estimate a drainage efficiency of the outflow pump based on at least the reservoir volume, the inflow rate, and the commanded outflow rate;
identify a drainage efficiency model from experimental or operational data to represent efficiency dynamics; and
apply a perfusion safety control routine that evaluates a safety barrier function based on the drainage efficiency model and adjusts circulation of perfusate between the inflow pump and the outflow pump based on the evaluated safety barrier function.
